(19) United States
(12) Patent Application Publication
(10) Pub. No.: US 2007/0043376 A1
(43) Pub. Date: Feb. 22, 2007

(54) Bone and cartilage implant delivery device

(75) Inventors: Neil C. Leatherbury, San Antonio, TX (US); Mark Q. Niederauer, San Antonio, TX (US); Fred B. Dinger III, San Antonio, TX (US); Joseph D. Blandford, San Antonio, TX (US)

Correspondence Address: GREENLEE WINNER AND SULLIVAN P C, 4875 PEARL EAST CIRCLE, SUITE 200, BOULDER, CO, 80301, US

(73) Assignee: OsteoBiologics, Inc., San Antonio, TX (US)

(21) Appl. No.: 11/292,807

(22) Filed: Dec. 2, 2005

Related U.S. Application Data
(63) Continuation-in-part of application No. 10/785,388, filed on Feb. 23, 2004.
(63) Continuation-in-part of application No. 11/290,142, filed on Nov. 30, 2005.
(60) Provisional application No. 60/448,965, filed on Feb. 21, 2003.
(60) Provisional application No. 60/632,050, filed on Nov. 30, 2004.

(51) Int. Cl. A61F 2/00

(52) U.S. Cl. 606/99

(57) ABSTRACT

A method and device for inserting an implant of synthetic material or healthy bone or cartilage into a bone or cartilage defect of unknown depth. The device includes an inner shaft within a hollow outer shaft. One end of the inner shaft of the device is suitable for inserting into the bone or cartilage defect in order to determine the depth, while the other end of the outer shaft is suitable for holding an implant. The implant is cut to fit the defect. The device is partially transparent or translucent to allow visualizing of the implant and defect. The delivery device can be bent or curved to allow the device to be introduced to a defect at different angles and positions. The methods and devices are suitable for delivery of implants to defects having complex shapes.

### CROSS-REFERENCE TO RELATED APPLICATIONS

This application is a continuation-in-part of pending U.S. application Ser. No. 10/785,388 filed Feb. 23, 2004, which in turn claims the benefit under 35 U.S.C. 119(e) of U.S. Provisional Application No. 60/448,965 filed Feb. 21, 2003; this application is also a continuation-in-part of pending a U.S. Application (attorney docket number 90-04 US) filed on Nov. 30, 2005, which in turn claims the benefit under 35 U.S.C. 119(e) of U.S. Provisional Application No. 60/632,050 filed Nov. 30, 2004, all of which are incorporated herein in their entirety to the extent not inconsistent herewith.

### BACKGROUND OF THE INVENTION

This invention relates to an apparatus and methods for performing repairs of cartilage and bone defects.

It is well known in the art that implants can be inserted into damaged bone or cartilage layers to treat injuries to those tissue layers. One type of implant procedure involves inserting plugs of healthy bone or cartilage that are harvested from a healthy area of the patient's body and transplanted into the defect, as disclosed in U.S. Pat. No. 5,152,763 (Johnson et al.), U.S. Pat. No. 5,919,196 (Bobic et al.), and U.S. Pat. No. 6,358,253 (Torrie et al.). In the alternative an implant can consist of synthetic material, such as porous biocompatible foams or polymers, for example as disclosed in U.S. Pat. No. 4,186,448 (Brekke et al.), U.S. Pat. No. 5,607,474 (Athanasiou et al.), and U.S. Pat. No. 5,716,413 (Walter et al).

In implant procedures, defects of variable depths are often presented. In order for the implant, once inserted into the defect, to evenly match the surface of the surrounding tissue without protruding or forming a cavity, the depth of the defect must be determined and the length of the implant tailored to fit the defect. Generally, it is difficult to determine the exact depth of a defect and, therefore, to insert an implant with the correct length.

Current devices for inserting implants, either bone or cartilage transplants or synthetic materials, are deficient in determining defect depth. U.S. Pat. No. 5,782,835 (Hart et al.) teaches a bone plug emplacement tool comprising a cylinder with an internal bore along the longitudinal axis and a stem disposed for co-axial movement within the internal bore. A bone plug placed in the internal bore is delivered into the defect when the stem is advanced through the bore. However, the tool does not provide means for determining the depth of the defect or for tailoring the length of the implant to fit the defect.

U.S. Pat. No. 6,395,011 (Johanson et al.) similarly teaches a device comprising a push rod within a hollow cylinder for harvesting and implanting bone plugs. In addition, the device includes a translucent or transparent tip permitting the surgeon to view the bone plug during implantation. Although this is an improvement in that it allows the length of the bone plug to be determined after harvesting, it also does not provide means to determine the depth of the defect.

Absent an implant delivery device with means for determining defect depth, other current methods of filling bone and/or cartilage defects include using a granular implant material to pack the defect, or using a separate plastic or metal depth gauge to measure the depth of the defect and then cutting the implant prior to insertion.

In the skeletal system, most of the major articulating joints, such as the knee or the hip, are comprised of relatively congruous surfaces which move smoothly through a range of motion. However, in certain articulating spaces, such as the ankle, the surfaces are comprised of more complicated geometries. For example, in the talus articulating surfaces are found on at least five surfaces. These articulating surfaces often converge in sharp transition points, creating a complicated geometry for surgical treatment in the event of acute or traumatic injury. Current therapies are usually limited to debridement, restricted motion, palliative drug therapy, osteochondral transplantation, or as a last resort, joint fusion. To recapitulate the articulating surface in an effort to reduce pain and restore function, the surgeon has few options. Currently, one common (although unpopular) option is to perform an osteochondral transplant from an articulating surface in the knee to the ankle. It is often difficult if not impossible to match the geometry between the donor and recipient surfaces, often resulting in marginal or unsatisfactory treatment. If the defect or injury is on the medial or lateral ridge of the talus, thus bridging two intersecting articular surfaces, there is no anatomical site from which a satisfactorily congruous donor tissue can be harvested.

A number of patents describe materials, devices and methods for cartilage repair which may be able to compensate for complex geometries. U.S. Pat. No. 5,716,413 (Walter et al.) describes moldable, hand-shapable biodegradable implant materials suitable for cartilage repair. U.S. Pat. No. 5,876,452 (Athanasiou et al.) describes biodegradable, porous, polymeric implant materials, and U.S. Pat. No. 6,511,511 (Slivka et al.) describes fiber-reinforced, porous, biodegradable implant devices suitable for cartilage repair.

Several patents also describe multi-phase materials or devices for repair to multiple tissues. U.S. Pat. No. 5,607,474 (Athanasiou et al.) describes a multi-phase bioerodible implant/carrier, including implants having a layer with properties similar to those of cartilage and a layer with properties similar to those of bone. U.S. Pat. No. 6,264,701 (Brekke) teaches devices having a first region with an internal three-dimensional architecture to approximate the histologic pattern of a first tissue; and a second region having an internal three-dimensional architecture to approximate the histologic pattern of a second tissue. U.S. Pat. No. 6,265,149 (Vyakarnam et al.) and U.S. Pat. No. 6,454,811 (Sherwood et al.) teach use of gradients in composition and/or microstructure and/or mechanical properties. U.S. Pat. Nos. 6,626,945 and 6,632,246 (Simon et al.) describe cartilage repair plugs having a composite structure. U.S. Pat. No. 6,626,945 (Simon et al.) teaches a variety of cartilage plug configurations, including two plug embodiments having an upper layer joining the plugs in which the upper surface of the upper layer is convex.

U.S. Pat. No. 6,358,253 (Torrie et al.) teaches methods for orienting a guide for use with surgical instruments perpendicular to a curved bone surface. In one configuration, the tissue-engaging portion of the guide is shaped so that a rim is formed above a flange. In use, the flange is seated in the bone and the rim contacts and is flush with the bone completely around its circumference. Torrie et al. also mention a configuration in which the tissue-engaging portion is in the form of an enlarged lip having a slightly concave surface. However, current devices for inserting tissue implants, such as bone or cartilage transplants, multi-phase materials, or other synthetic materials, are deficient for inserting implants in complex surfaces which are not planar or smoothly curved.

There remains a need in the art for improved implants, surgical equipment, and repair methods for defects in bone and cartilage tissue having an unspecified depth and a nonplanar or complex surface.

### SUMMARY OF THE INVENTION

The present invention provides a bone and/or cartilage implant delivery tool, which allows for measuring, sizing, and delivering of an implant to a bone and/or cartilage defect of unknown depth. Defects are not limited to bone and cartilage injuries. Defects can be intentionally created, such as the hole remaining in bone or cartilage tissue after a plug of healthy bone or cartilage is removed for transplantation. Intentionally created defects also include holes in bone or cartilage tissue created in order to insert autologous or allogenic grafts during ligament or tendon repair surgeries. This device is useful for arthroscopic repair of an osteochondral defect in a joint, such as a knee, and is also suitable for treatment of any bone or cartilage defect that is accessible by the device. Furthermore, the device is suitable for use with bone and cartilage transplants as well as synthetic implants. As used herein, “implant” includes implants made from synthetic materials and implants that are bone and cartilage transplants.

The delivery device of the present invention includes a tubular outer shaft having a proximal and a distal end and an internal bore along the longitudinal axis. In the present context, “proximal” refers to the end of the device initially oriented closest to the patient's body and used in measuring the depth of the defect as described below. “Distal” refers to the end of the device initially oriented away from the patient's body and used to contain the implant. The internal bore of the outer shaft is sized to accommodate the diameter of the implant or the profile of the implant if the implant is non-cylindrical.

A cylindrical inner shaft, also having proximal and distal ends, is disposed within the internal bore in the outer shaft, wherein the proximal end of the inner shaft is suitable for insertion into a defect. By “suitable for insertion into a defect” it meant that the proximal end of the inner shaft has a size and shape allowing it to fit within a bone and/or cartilage defect without distorting the defect or damaging the tissue layers. In one embodiment of the present invention, the proximal end of the inner shaft has a size and shape similar to the size and shape of the implant. The inner shaft has a diameter that also allows it to be slidably engaged with the outer shaft. “Slidably engaged” means the inner shaft can slide within the bore in the outer shaft. The inner shaft may be solid or have a cannula through its center. The delivery device may further comprise a guide wire disposed in the cannula, where one end of the guide wire is attached to a defect and the other end of the guide wire passes through the distal end of the inner shaft and extends to the proximal end of the inner shaft.

The delivery device comprises means to provide friction-retarded movement of the inner shaft through the outer shaft. The inner shaft may have a “friction member”, which is herein defined as a section of the inner shaft having a diameter large enough to contact the inner surface of the outer shaft and provide a tight fit within the internal bore. The friction member may be coated with rubber or other materials to provide additional friction. The surfaces of the outer shaft and inner shaft also may be modified to provide friction-retarded movement. For example, a section of the outer shaft's inner surface may contain small beads and a corresponding section of the inner shaft's outer surface may contain small ridges. When the inner shaft is moved through the outer shaft, the small beads on the outer shaft contact the ridges on the inner shaft and provide additional friction. Alternatively, a section on the inner surface of the outer shaft may contain ridges or serrated teeth that engage ridges or serrated teeth disposed on the corresponding section on the outer surface of the inner shaft. When the inner shaft is moved through the outer shaft, the ridges and/or serrated teeth contact each other and movement is restricted. Other means that prevent unwanted movement of the inner shaft through the outer shaft include otherwise texturing the surfaces of the inner shaft and outer shaft, or coating the surfaces of the inner shaft and outer shaft with a viscous liquid.

In addition, the delivery device may be designed to limit rotation of the inner shaft within the outer shaft. For example, one of a key or keyway may be located on the inner shaft, with the other of key or keyway located on the outer shaft. The interlocking of the key and keyway limits or prevents rotation of the inner shaft within the outer shaft. Configurations other than a key and keyway can act to limit rotation of the inner shaft within the outer shaft. As a simple example, rotation of the inner shaft within the outer shaft can be limited if both have square or rectangular cross-sections and the inner shaft fits closely within the outer shaft.

When the inner shaft is disposed in the outer shaft so that the inner shaft does not protrude from the proximal end of the outer shaft, inserting an implant into the distal end of the outer shaft displaces the inner shaft towards the proximal end causing a portion of the inner shaft to protrude from the proximal end of the outer shaft. Conversely, when an implant is preloaded into the distal end of the outer shaft, the inner shaft is inserted in the proximal end of the outer shaft and advanced toward the distal end of the outer shaft until the distal end of the inner shaft contacts the implant. At this point, the implant will not extend beyond the distal end of the outer shaft and a portion of the inner shaft will protrude from the proximal end of the outer shaft.

With an implant at least partially inserted into the distal end of the outer shaft, the proximal end of the inner shaft is inserted into a defect of unknown depth. When the proximal end of the inner shaft contacts the bottom of the defect, the outer shaft is advanced towards the defect until the proximal end of the outer shaft contacts the surface of the tissue surrounding the defect. In relation to the outer shaft, this motion distally advances the inner shaft. As a result, the length of the inner shaft that protrudes from the proximal end of the outer shaft equals the depth of the defect. In addition, this motion displaces the implant in the outer shaft and causes a portion of the implant to extend beyond the distal end of the outer shaft.

The protruding end of the implant, i.e., the portion of the implant protruding from the distal end of the outer shaft, can be cut off with a knife or other cutting device. The remaining length of the implant in the distal end of the outer shaft equals the length of the inner shaft that protrudes from the proximal end of the outer shaft, which also equals the depth of the defect. The proximal end of the device is removed from the defect and the distal end of the device containing the implant is placed over the defect. The proximal end of the inner shaft, which is now the end furthest from the patient's body, is advanced towards the distal end of the outer shaft, which is now the end closest to the patient's body, pushing the implant into the defect.

If there is an unobstructed path to the defect, the delivery device is inserted over the defect so that the delivery device is perpendicular to the tissue surface surrounding the defect. However, for some joints, such as the knee or elbow, a perpendicular approach is not available. Surrounding bone, cartilage, ligament, tendon or other tissue prevent easy access to the defect, requiring more invasive procedures, such as surgery, to gain access to the defect, or resulting in improperly inserted implants. In one embodiment of the invention, the delivery device is curved or bent at an angle near or at the distal end of the outer shaft. Thus, the delivery device does not have to be perpendicular to the tissue surrounding the defect to deliver the implant. The delivery device is advanced toward the defect at an angle until the distal end of the outer shaft is aligned over the defect. The curve or bend can be placed at the distal most end of the outer shaft or at a more proximal position depending on which design provides easier access for the given defect. The inner shaft is constructed from a flexible material or with a design that allows it to advance through the curve or bend and push the implant into the defect. A flexible inner shaft includes, but is not limited to, (1) a pliable material such as a rubber or plastic where the flexibility is inherent in the mechanical properties of the material, (2) a rigid thin walled material that is coiled, like a spring, or (3) a thin walled tube that is contains a continuous spinal cut. The angle of the curve or bend can be any angle that still permits the inner shaft to advance through the outer shaft.

Often times, it is not possible to know what angle is available for the delivery device to approach the defect. In one embodiment, the distal end of the outer shaft is made from a semi-flexible material or contains a hinge or a plethora of hinges to allow the device to change its angle along the body. This allows the distal end of the delivery device to be bent into various positions. In this embodiment, the defect can be approached from a wide range of angles and directions. In a further embodiment, the flexible tip is constructed from a material, such as a rubber or plastic where the flexibility is inherent in the mechanical properties of the material. The distal end of the outer shaft is flexible enough so that the angle and orientation of the distal end can be adjusted by hand, but is rigid enough so that it retains its shape while the delivery device is being positioned over the defect.

A further embodiment of this invention includes the proximal and distal ends of the device having smooth, rounded edges to prevent damaging surrounding tissues. While the device can be constructed of any materials, including, but not limited to, medical grade plastic or metal, it is preferred that plastic is used to prevent scratching the bone or cartilage surface. In a further embodiment, a series of thin concentric slots cut into the outer surface of the outer shaft provide a gripping surface for easier handling of the device.

A further embodiment of this invention includes at least one slot or window in the distal end of the outer shaft of the device for visualizing the implant. The slot or window may be of any shape that allows the implant to be seen while the implant is disposed within the delivery device. The slot or window can also be covered with transparent material.

In another embodiment of this invention, the device is made, partially or entirely, from translucent or, more preferably, transparent material to allow visualization of the implant or defect. By “translucent” it is meant that light is transmitted through the material so that the implant is visible while disposed in the outer shaft but with some loss in clarity. By “transparent” it is meant that light is transmitted through the material with little to no loss in optical clarity. Translucent and transparent materials suitable for this embodiment are known in the art and include, but are not limited to: polycarbonate, such as the Lexan® series of resins (GE Plastics); acrylonitrile butadiene styrene, such as Cycolac® CTS-100 and CTR52F (GE Plastics); and polypropylene, such as resin #4018 (Amoco). Using such materials, the entire device or a portion of the device housing the implant can be made to be transparent or at least translucent. In one embodiment, the entire outer shaft is translucent or transparent while the inner shaft remains opaque. In another embodiment, only the distal end, or at least part of the distal end, of the outer shaft is translucent or transparent. Alternatively, the outer shaft is opaque except for one or more sections at the distal end which are translucent or transparent. The inner shaft is optionally color coded to provide easy identification of the device and to correspond to a specific size of the internal bore. In another embodiment, the translucent or transparent material is tinted with a color so that it remains at least translucent but so that the color is noticeable. The color of the translucent or transparent material provides easy identification of the device and corresponds to a specific size of the internal bore.

In a further embodiment of this invention, the distal end of the outer shaft is tapered inward, creating slight compression on the implant to prevent undesired movement of the implant within the device. Alternatively, the outer shaft includes tapered leaves in the distal end of the outer shaft. Longitudinal slots are cut in the distal end of the outer shaft, creating opposing leaves. The leaves are the sections of the outer shaft between the longitudinal slots. These leaves can be made to taper slightly inward, creating slight compression on the implant.

A further embodiment of this invention includes a snap-bead feature on the distal end of the outer shaft for attaching items to the device. The snap-bead feature comprises an annular groove around the distal end of the outer shaft. An attachable item has one or more small beads or a rim that fits into this groove. One such attachable item is a temporary cap that fits over the distal end of the outer shaft to prevent accidental removal of the implant from the device.

When the implant is delivered to the bone or cartilage defect, the delivery device will often pass through soft tissue. In order to pass through soft tissue more easily and without disrupting the implant, a removable outer sleeve having a bulleted tip is disposed over the outer shaft of a delivery device. Once the delivery device is introduced to the defect, the sleeve may be removed and retracted. Preferably, the removable outer sleeve is clear to allow visualization of the delivery device and the defect. Alternatively, the delivery device has threading on the outside of the outer shaft allowing the device to be twisted into the soft tissue to make insertion easier.

In a further embodiment of this invention, the implant is delivered to a defect with bioactive fluids, such as blood, blood concentrate or cell suspension. After the implant has been sized and cut to fit the defect, a cap will be placed around the distal end of the outer shaft and bioactive fluids added via a window or slot. Additionally, a centrifuge can be used to load fluids and the delivery device can be made suitable for use in a centrifuge, i.e., structurally able to withstand the forces during centrifugation without leaking or damaging the implant, when loading fluids to the implant.

Defects may occur such that the shape of the tissue surface at the defect area is complex. For example, it may be desirable to place an implant along a ridge between two articulating surfaces. The present invention also provides methods for delivering the implants with a complex proximal surface in bone and/or cartilage tissue. With reference to an implant, the “proximal surface” refers to the surface of the implant which, when inserted in the tissue defect, will be closest to the surface of the surrounding tissue. The proximal surface of the implant is designed to be a clinically acceptable replacement for tissue at the defect site. The proximal surface of the implant is also congruous with the tissue which surrounds the implant once it is implanted.

In one embodiment, the proximal surface of the implant comprises two facets converging to form an angled surface. Such an implant can be used to match converging articular surfaces in the talus, typically the talar dome and surfaces which articulate with either the medial or lateral malleolus. In other embodiments, the proximal surface of the implant can be concave or convex. Another application where an implant with a complex articulating surface can be used to restore anatomical function is in the knee. For example, the implants of the invention can be used in the trochlea, the patella, or the patello-femoral joint. The implant could be constructed with a concave shape to match the trochlear sulcus of the femur. Similarly, the implant could be fabricated with a convex, slightly rounded surface to match the surface of the patella.

Still another example of a complex geometry where an implant with a complex surface would be useful is the small joints of the hands and feet. For example, the carpometacarpal tarsal joints, and metatarsal joints (including metatarsal head joints) represent complex, highly curved surfaces that require implants with complex geometries. Other examples of joints suitable for the implants of the present invention include the temporomandibular joint (TMJ) of the jaw bone, spine joints (including vertebra and facet joint), hip, shoulder, and elbow.

In one embodiment of the present invention, the delivery device contains indentations or notches at the distal and proximal end of the outer shaft that conform to the surface of the tissue surrounding the defect, and the distal end of the inner shaft contains an indentation or notch that conforms to the proximal surface of the implant. Preferably, the proximal surface of the implant matches the contours of the of the tissue surface surrounding the defect. When the distal end or proximal end of the outer shaft is placed over a defect at a complex surface, the indentations or notches allow the outer shaft to better fit over the tissue surface containing the defect. Likewise, the indentation or notch at the distal end of the inner shaft allows for a better fit with the proximal end of an implant having a complex shape, and allows for an even distribution of pressure as the inner shaft pushes the implant into the defect.

In one embodiment, the implant delivered to the defect is a synthetic implant. The implant may be a single or multi-phase construct. A dual phase implant can be used to simulate a combination of cartilage and bone. A multi-phase implant with three phases could be used to simulate a surface with three adjacent tissues, such as articular cartilage, cancellous bone, and cortical bone. Such an implant could be useful in reconstructing a damaged femoral or tibial epiphysis. The various layers may be separated by a non-permeable film to isolate the different portions of the multiphase implant construct.

This invention also includes a cutting device comprising a cutting base having a hole adapted for receiving an implant protruding from the outer shaft of the implant delivery device and may also comprise at least one cutting blade for cutting off the portion of the implant that protrudes from the distal end of the outer shaft. “Adapted for receiving an implant” or “adapted for receiving the protruding end of an implant” with respect to the hole in the cutting base means the hole is big enough to allow the protruding end of the implant to pass through the hole, but at some point is small enough to prevent the distal end of the outer shaft from passing further through the hole. The point at which the hole allows the protruding end of the implant, but not the distal end of the outer shaft, to pass through is where the implant is cut. This point may be along the top or bottom surface of the cutting device base or somewhere within the cutting device base.

One embodiment of the implant cutting device comprises: a base comprising a vertical hole therethrough for receiving the protruding end of an implant and means for receiving at least one cutting blade; and at least one cutting blade adapted to slide within said means for receiving at least one cutting blade and cut off the protruding end of the implant. The “means for receiving a cutting blade” include a horizontal slot through the cutting device base or guides along the top or bottom surface of the base that allow the cutting blade to intersect the hole at the point where the implant but not the outer shaft can advance through the hole. The device may include a plurality (two or more) of cutting blades.

This invention also includes an implant capsule loader for inserting an implant into the shaft of an implant delivery device for delivery and orientation of multiple implants. The capsule loader comprises a hollow tube having a front end and a back end, adapted to fit within the distal end of the outer shaft of an implant delivery device. The capsule loader may also comprise a backplate disposed within said hollow tube covering the opening at the back end of said tube; and at least one flexible leaflet along the outer surface of said hollow tube fixed at the front end of said hollow tube and having a free end toward the back end of said hollow tube, said flexible leaflet having an outwardly extending prong at the free end thereof; said prong being adapted to fit within a hole in said shaft.

The terms “tube”, “tubular” and “cylindrical” used to describe the implant delivery device and implant capsule loader do not exclude depressions, reliefs, flats or flutes, or limit the shapes to only round cylinders. A tube is a hollow conduit, the cross-sectional area of which need not be circular or uniform along the length of the tube. The cross-sectional area of a tube can be any shape including, but not limited to, elliptical, hexagonal, octagonal, or irregular.

This invention also includes a kit comprising at least one implant delivery device. The kit may also include an implant and a knife or cutting device. The kit may comprise several implant delivery devices having different sizes of internal bores and inner shafts in order to accommodate defects and implants of varying sizes. The delivery devices of this kit can be individually color coded according to size. The invention also provides apparatus, kits and methods for creation of a defect having a selected location, diameter and depth in tissue having a nonplanar or complex surface. The apparatus and methods create defects which are compatible with the plug implants of the invention.

### BRIEF DESCRIPTION OF THE DRAWINGS

FIG. 1 shows an implant delivery device of this invention with the inner shaft protruding from the proximal end of an outer shaft.

FIG. 2 shows the inner shaft of an implant delivery device of this invention.

FIG. 3 shows the outer shaft of an implant delivery device of this invention.

FIG. 4 shows a cross-sectional view of the implant delivery device of FIG. 1.

FIG. 5 shows an implant delivery device of this invention having longitudinal slots and a snap-bead feature on the distal end of the outer shaft with an inner shaft protruding from the proximal end of an outer shaft.

FIG. 6 shows the implant delivery device of FIG. 5 with an uncut implant disposed in the distal end of the outer shaft.

FIG. 7A is a cross-sectional side view of a cutting device of this invention with the distal end of the implant delivery device placed in the vertical hole therein. FIG. 7B is an exploded assembly view of the cutting device, also showing the distal end of the implant delivery device.

FIG. 8A is an end view of the inner shaft of the implant delivery device of FIG. 5 comprising a cannula. FIG. 8B is a side view of an inner shaft having ridges. FIG. 8C is an expanded view of the circled section of FIG. 8B showing the ridges in greater detail. The cannula in FIGS. 8B and 8C is shown by dotted lines.

FIG. 9A is an end view of the outer shaft of the implant delivery device of FIG. 5. FIG. 9B is a cross-sectional side view of the outer shaft shown in FIG. 9A. FIG. 9C is an expanded view of the circled section of FIG. 9B showing friction beads on the inner surface of the outer shaft.

FIG. 10A is an end view of a modified inner shaft of the implant delivery device of FIG. 5 comprising two alignment ribs. FIG. 10B is a side view of a modified inner shaft. FIG. 10C is an expanded view of the circled section of FIG. 10B showing serrated teeth along the surface of the inner shaft. The cannula in FIGS. 10B and 10C is shown by dotted lines.

FIG. 11A is an end view of a modified outer shaft of the implant delivery device of FIG. 5 comprising alignment slots. FIG. 11B is a cross-sectional side view of a modified outer shaft. FIG. 11C is an expanded view of the circled section of FIG. 11B showing serrated teeth on the inner surface of the outer shaft.

FIG. 12A shows cross-sectional view of an implant capsule loader containing an implant. The capsule loader is disposed within the outer shaft of the implant delivery device of FIG. 5. FIG. 12B shows an external view of an implant capsule loader of this invention. FIG. 12C shows a cross-sectional view of a capsule loader with the outer shaft of the implant delivery device after the inner shaft has pushed the implant out of the capsule loader and delivery device.

FIG. 13 shows the inner shaft and outer shaft of an implant delivery device of this invention, where the distal end of the inner shaft and outer shaft are essentially flat.

FIG. 14 shows the inner shaft and outer shaft of an implant delivery device similar to that depicted in FIG. 13, but where the distal end of the inner shaft and outer shaft have indentations or notches that are contoured to have a shape corresponding to the tissue surface surrounding the defect.

FIG. 15 shows the outer shaft of an implant delivery device of the present invention having a tapered tip.

FIG. 16 shows a delivery device of the present invention having a threaded outer surface.

FIG. 17 shows the outer shaft of a delivery device of the present invention where the distal end of the outer shaft is bent at an angle.

FIG. 18 shows the outer shaft of a delivery device of the present invention where the distal end of the outer shaft is curved at an angle.

FIG. 19 shows a delivery device of the present invention where the distal end of the outer shaft is flexible and can be shaped into different angles and positions.

FIG. 20 shows a removable outer sleeve disposed over the outer shaft of a delivery device of the present invention.

FIG. 21 shows a top view of the outer shaft of FIG. 14 having indentations or notches at the distal and proximal ends.

FIG. 22A and FIG. 22B show a delivery device having indentations or notches at the distal end of the outer shaft placed in contact with a ridged tissue surface.

### DETAILED DESCRIPTION OF THE INVENTION

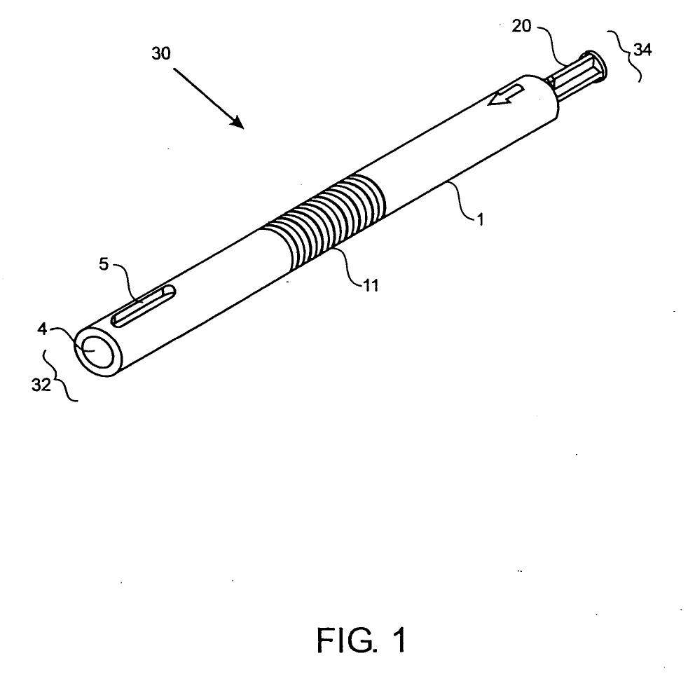

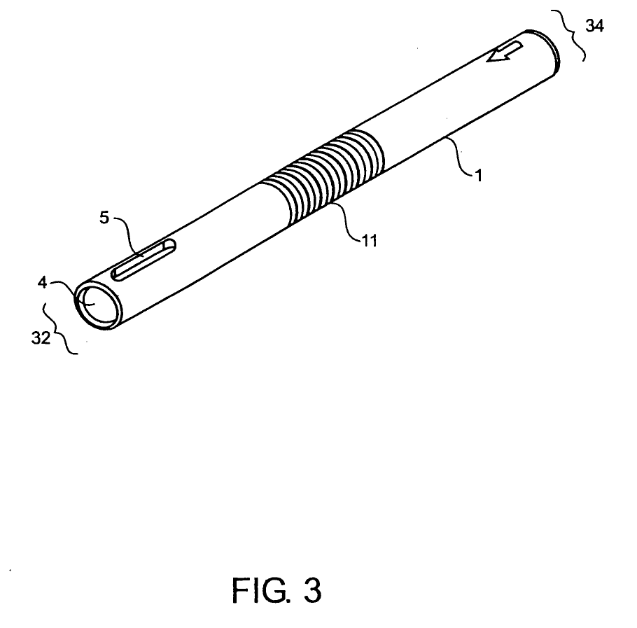

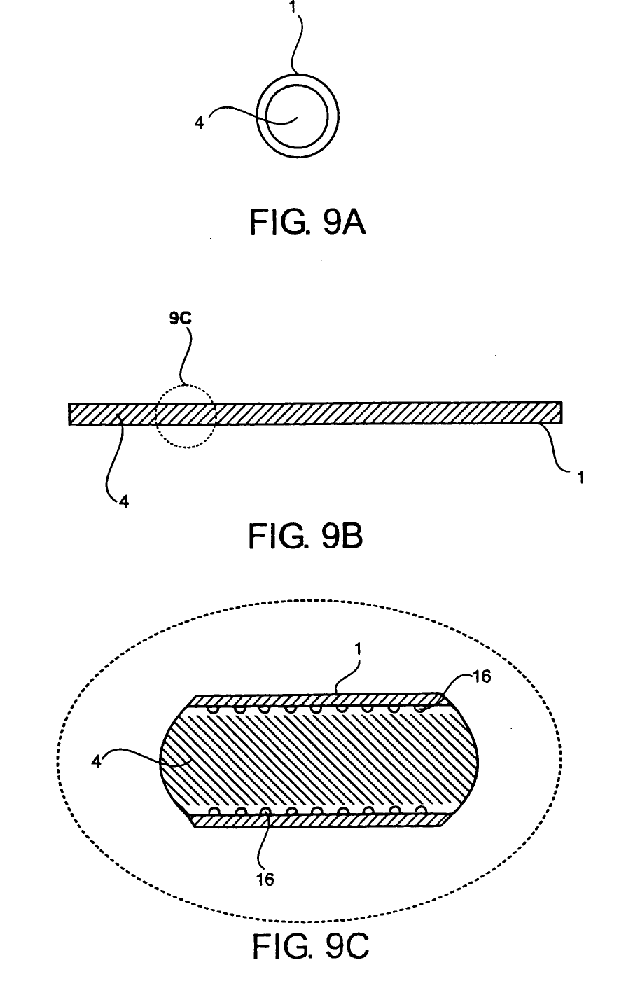

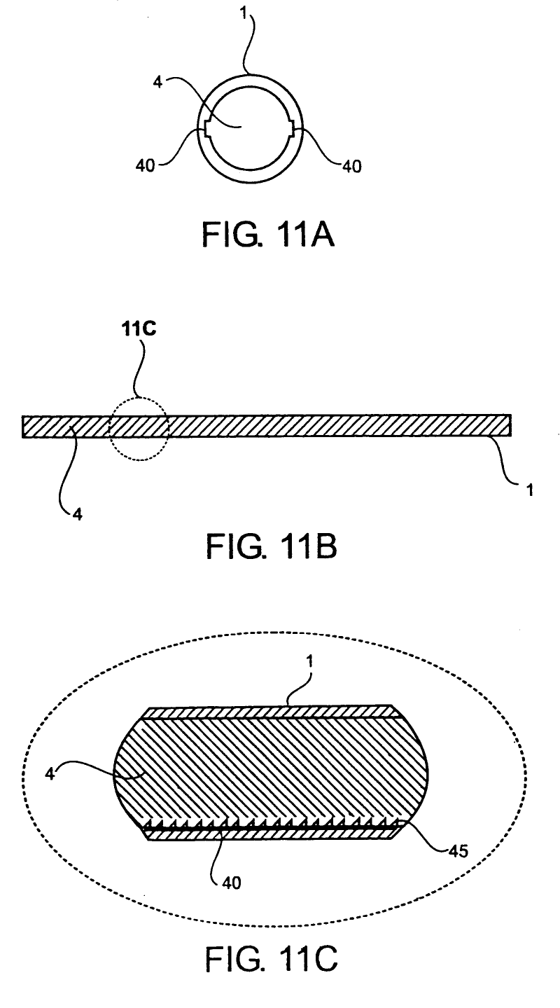

FIG. 1 shows one embodiment of the implant delivery device 30 of the present invention having a proximal end 34 and a distal end 32. In a preferred embodiment, the delivery device 30 has a length suitable for arthroscopic use, i.e., approximately 4 to 10 inches long, preferably 5-8 inches, with a diameter of about 0.25-1 inch, preferably 0.4-0.75 inches. The implant delivery device 30 includes a hollow tubular outer shaft 1 (also shown in FIG. 3) having an internal bore 4 along the longitudinal axis. The internal bore 4 extends the entire length of the outer shaft 1 from the distal end 32 to the proximal end 34. FIGS. 9A-9C and FIGS. 11A-11C also illustrate the internal bore 4. The distal end 32 of the outer shaft 1 can have one or more slots 5 through the outer shaft 1 for visualizing the implant (not shown in FIG. 1) when the implant is in the delivery device 30. Slots 5 can be any shape that allows the implant to be visualized while disposed in the delivery device 30 and can be covered with transparent material. Alternatively, the entire outer shaft 1 or the distal end 32 of the outer shaft 1 may be transparent or translucent to allow the implant to be visualized while disposed in the delivery device 30. The outer shaft 1 optionally contains a gripping surface 11, which is a series of thin concentric slots cut into the outer surface of the outer shaft. The gripping surface 11 may be located anywhere along the length of the outer shaft 1.

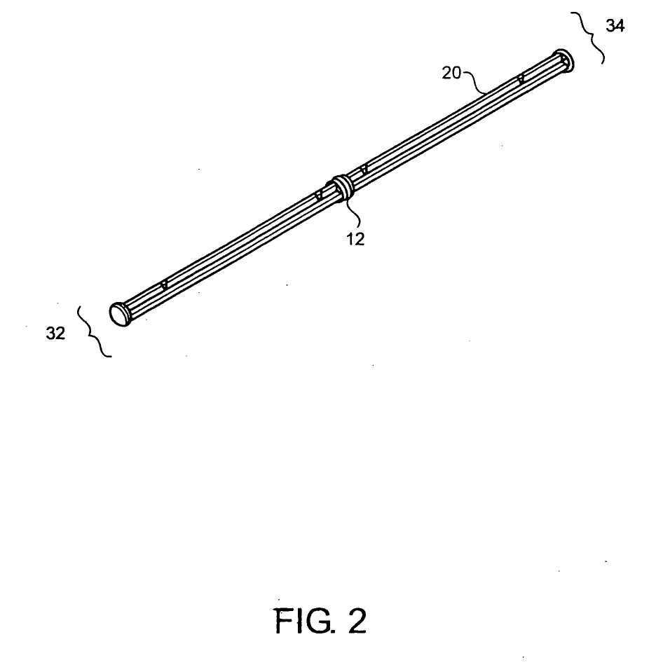

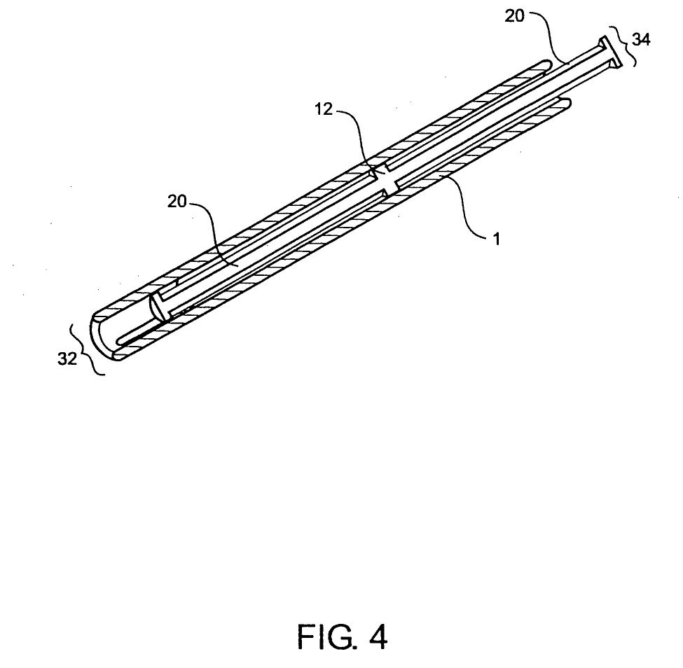

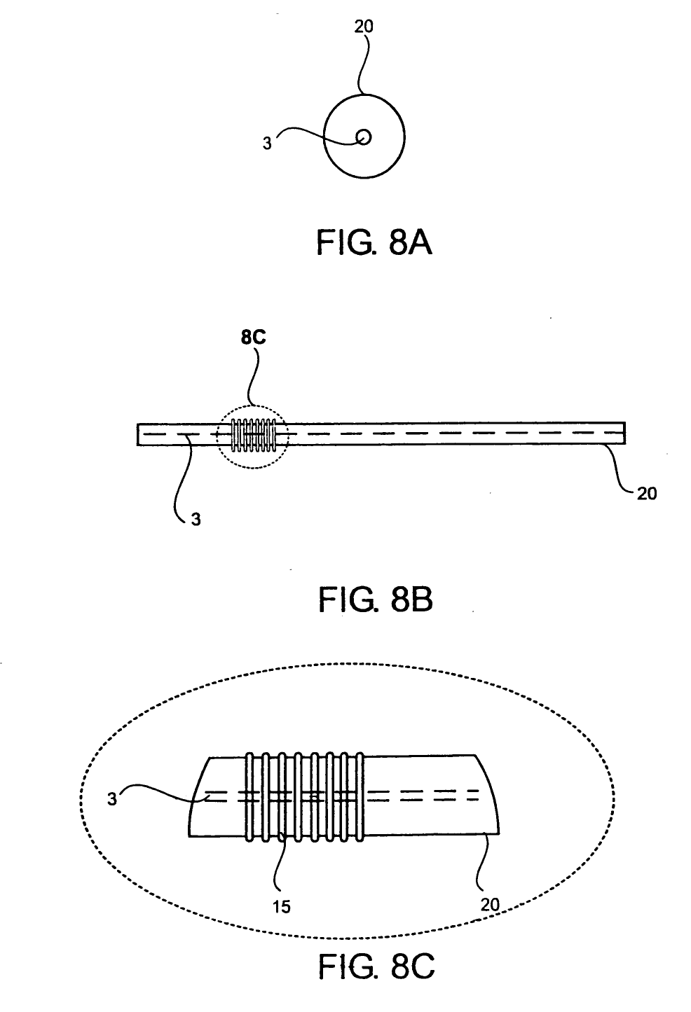

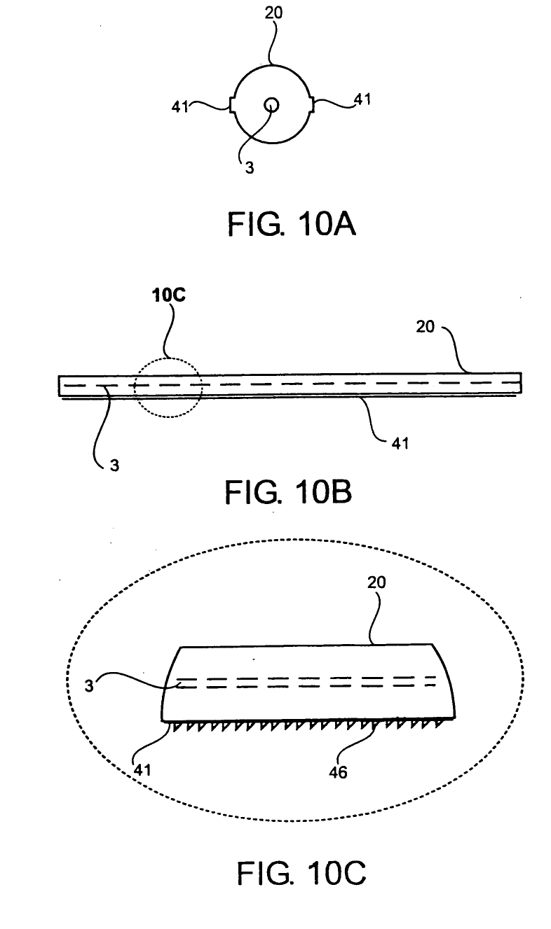

The delivery device 30 illustrated in FIG. 1 further comprises an inner shaft 20 also having proximal and distal ends. The inner shaft 20 is situated within the outer shaft 1 and is able to move proximally and distally through the internal bore 4. FIG. 4 shows a cross-section of delivery device 30 with the inner shaft 20 disposed within the internal bore 4 of the outer shaft 1. As shown in FIGS. 2 and 4, inner shaft 20 has a friction member 12 which contacts the inner surface of the outer shaft 1. Optionally, the inner shaft may contain a small cannula 3 through its center, as shown in FIGS. 8A-8C and 10A-10C. A guide wire attached to the defect by a means such as suturing may be threaded through cannula 3. Optionally, the inner shaft 20 may also be transparent or translucent.

In some embodiments, a guide wire, such as a Kirschner wire (K-wire), is used to insert the implant into the defect. The K-wire can be attached to the defect during the creation of the defect, or the K-wire can be attached afterwards, such as by suturing. When a K-wire is used to guide the implant into the defect, a delivery device 30 is used having a cannula 3 in the inner shaft 20, the cannula 3 being sized to permit passage of the guide wire. Also, an implant with a central hole to permit passage of the guide wire can be used under these circumstances. The K-wire is threaded through the implant (not shown) and inner shaft 20 thereby aligning the delivery device and implant with the defect. In this embodiment, the K-wire preferably has a diameter of approximately 1.0-2.0 mm, more preferably 1.5 mm.

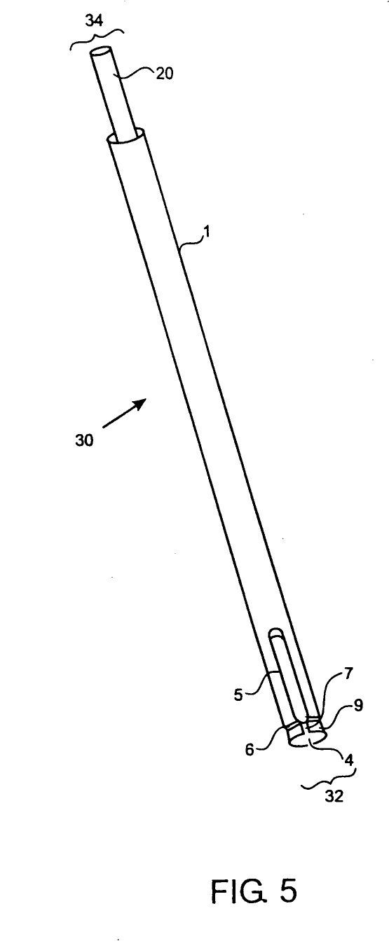

FIG. 5 shows another embodiment of the present invention where the distal end 32 of the delivery device 30 has a small groove 6 running around the outside of the outer shaft 1. In this embodiment, items can attach to the distal end 32 of the outer shaft 1 by having a diameter slightly larger than the outer diameter of the outer shaft 1, fitting over the distal end 32 of the outer shaft 1, and having one or more beads or a rim that snap into the groove 6, thus securing the position of the attached item.

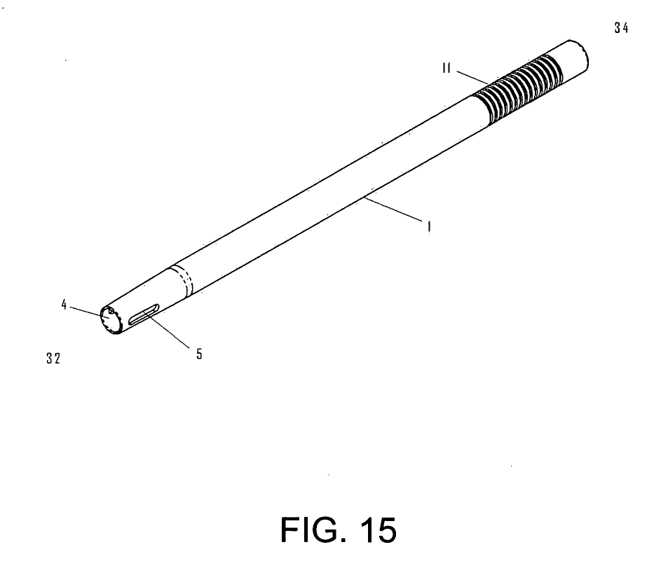

FIG. 5 also shows the delivery device 30 having thin longitudinal slits 7 cut through the distal end 32 of the outer shaft 1 creating leaves 9. Leaves 9 are the sections of the outer shaft 1 between the longitudinal slits 7. The leaves 9 can be made so that they taper slightly inward creating slight compression on the implant (not shown) while in the device 30. Alternatively, as depicted in FIG. 15, the distal end 32 of the outer shaft 1 can be tapered inward without the use of leaves to create slight compression on the implant. FIG. 15 also shows the gripping surface 11 located closer to the proximal end 34 of the outer shaft 1.

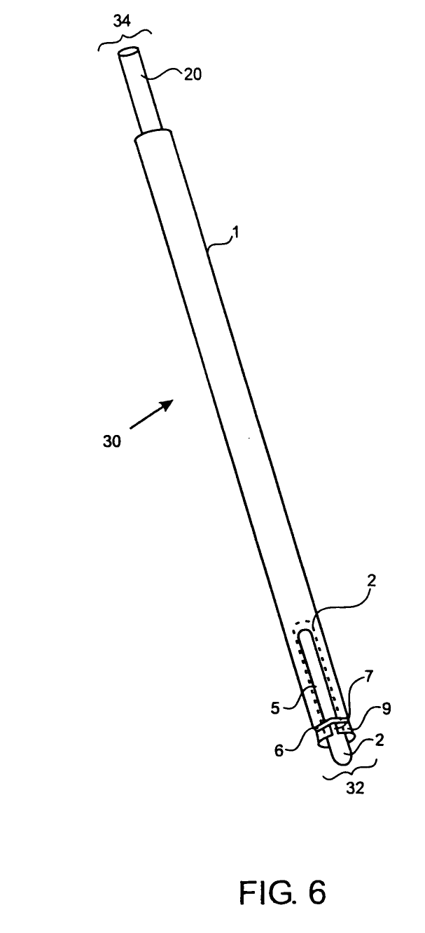

FIG. 6 shows the implant delivery device 30 illustrated in FIG. 5 with an implant 2 disposed in the distal end 32 of the outer shaft 1. In this figure, a portion of the implant 2 extends beyond the distal end 32 of the outer shaft 1 and would have to be cut.

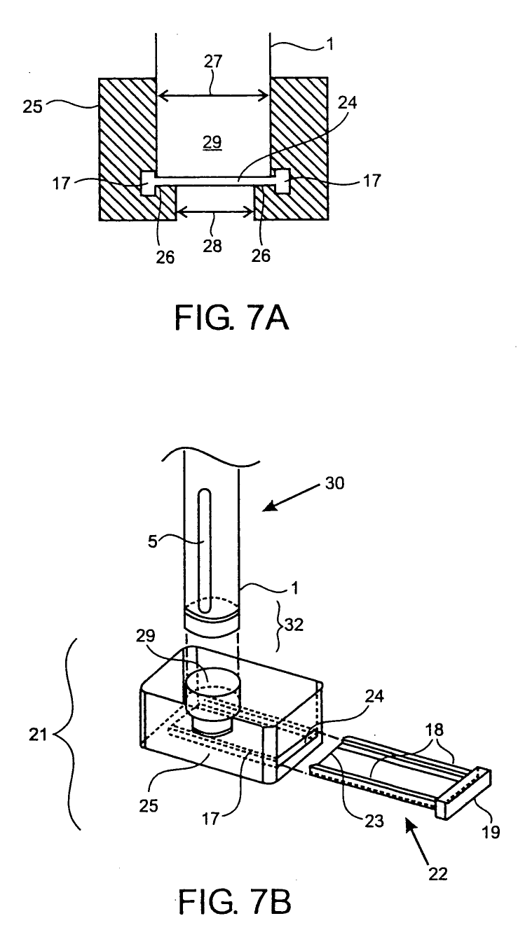

FIGS. 7A and 7B show a preferred embodiment of a cutting device 21 comprising a rectangular base 25 and a cutting blade 22. Rectangular base 25 has a vertical circular hole 29 extending through the base 25 from top to bottom, having an upper diameter 27 and lower diameter 28. The upper diameter 27 is slightly larger than the outer diameter of the outer shaft 1 of the device 30. The lower diameter 28 is slightly less than the outer diameter of the outer shaft 1 but slightly larger than the diameter of implant 2 shown in FIG. 6. Within the hole 29, a shoulder 26 is formed where the upper diameter 27 meets the lower diameter 28. A cutting slot 24 horizontally extends from one side of base 25 and perpendicularly intersects hole 29 at shoulder 26. The sides of cutting slot 24 vertically expand into guide slots 17.

A cutting blade 22 with a sharp cutting edge 23 fits within the cutting slot 24 and can be advanced through cutting slot 24 until the cutting edge 23 is completely advanced across the hole 29. Opposite and parallel to cutting edge 23, cutting blade 22 has a handle edge 19, which has a greater height and width than cutting edge 23. Handle edge 19 is not sharp and is suitable for holding onto by hand. Cutting blade 22 also has two guide edges 18, which intersect and extend from cutting edge 23 to handle edge 19. Guide edges 18 have a greater height than cutting edge 23 and fit into guide slots 17 to provide a secure insertion of cutting blade 22 into cutting slot 24.

To use the implant delivery device 30 in one embodiment of the present invention, the inner shaft 20 is placed within the internal bore 4 of the outer shaft 1 so that no portion of the inner shaft 20 protrudes from the outer shaft 1. An implant 2, which can be a synthetic implant or a transplant of healthy bone or cartilage, is inserted into the distal end 32 of the outer shaft 1. This pushes inner shaft 20 through internal bore 4 toward proximal end 34. As a result, a portion of inner shaft 20 will protrude from proximal end 34 of outer shaft 1. The portion of inner shaft 20 that protrudes from proximal end 34 of outer shaft 1 will be the same length as implant 2 within distal end 32 of outer shaft 1.

The portion of inner shaft 20 that protrudes from the proximal end 34 of the outer shaft is then inserted into a defect. When the proximal end 34 of the inner shaft 20 contacts the bottom of the defect, outer shaft 1 is proximally advanced until the proximal end 34 of the outer shaft 1, which has a larger diameter than inner shaft 20 and the defect, is level with and contacts the surface of the tissue surrounding the defect. This act displaces inner shaft 20 through internal bore 4 toward distal end 32 of outer shaft 1, causing a portion of the implant 2 to extend beyond the distal end 32 of outer shaft 1.

The protruding end of implant 2, i.e., the portion of implant 2 extending beyond the distal end 32 of the outer shaft 1, is then cut off. In one embodiment, a knife is used to cut implant 2. In another embodiment, the cutting device 21 illustrated in FIGS. 7A and 7B is used. To use cutting device 21, the distal end 32 of outer shaft 1 is inserted through vertical hole 29 in base 25 until outer shaft 1 contacts shoulder 26. The shoulder 26 prevents outer shaft 1 from advancing further through hole 29, but because the lower diameter 28 is equal to or slightly larger than the diameter of internal bore 4, the portion of implant 2 that extends beyond the distal end 32 of the outer shaft 1 passes through vertical hole 29 beyond the shoulder 26. Cutting blade 22 is inserted into cutting slot 24 and advanced until cutting edge 23 horizontally intersects vertical hole 29 and cuts through implant 2. The cutting device 21 is removed after cutting off the protruding portion of the implant.

The device 30 can be removed from the defect prior to or immediately after cutting off the excess implant material. Once removed from the defect, implant delivery device 30 is flipped around so that the distal end 32 of the device 30 is oriented toward the defect. The distal end 32 of outer shaft 1 is placed over the defect. In embodiments having a slot 5 or where the device 30 is made from translucent or transparent materials, the implant 2 can be visualized allowing the device 30 to be orientated so that the implant 2 is placed in the desired position in relation to the defect. The inner shaft 20 is advanced through the internal bore 4 towards distal end 32, pushing the remaining portion of implant 2 into the defect. The defect, if intentionally created, is formed with a diameter such that implant 2 completely fills the defect.

Another embodiment (not shown) of cutting device 21 comprises hole 29 having a diameter slightly less than the outer diameter of outer shaft 1 but slightly larger than the diameter of implant 2. In this embodiment, the portion of implant 2 that extends beyond the distal end 32 of outer shaft 1 can be inserted into hole 29 but the distal end 32 of outer shaft 1 cannot be inserted into hole 29. Guide slots 17 are disposed into the top surface of base 25. Guide edges 18 of cutting blade 22 fit into guide slots 17 allowing cutting blade 22 to slide along the top surface of base 25 until cutting edge 23 cuts through implant 2 at the top of hole 29.

Another embodiment (not shown) of cutting device 21 comprises hole 29 having a diameter slightly larger than the outer diameter of outer shaft 1 until hole 29 reaches the bottom surface of base 25. At the bottom surface of base 25, hole 29 has a diameter slightly less than the outer diameter of outer shaft 1 but slightly larger than the diameter of implant 2. In this embodiment, the portion of implant 2 that extends beyond the distal end 32 of outer shaft 1 can exit through the bottom of hole 29 but the distal end 32 of outer shaft 1 cannot. Guide slots 17 are disposed into the bottom surface of base 25. Guide edges 18 of cutting blade 22 fit into guide slots 17 allowing cutting blade 22 to slide along the bottom surface of base 25 until cutting edge 23 cuts through implant 2 at the bottom of hole 29.

FIGS. 8A-8C show an embodiment of this invention wherein a section of inner shaft 20 comprises ridges 15. Ridges 15 are raised rings around a portion of the outer surface of inner shaft 20. In this embodiment, friction beads 16 are also disposed on the corresponding section of the inner surface of outer shaft 1, as shown in FIGS. 9A-9C. The friction beads 16 are raised higher than the surrounding inner surface of outer shaft 1. During proximal and distal movement of inner shaft 20 through internal bore 4 of outer shaft 1, friction beads 16 engage with ridges 15 requiring extra force to continue to advance the inner shaft 20 through the internal bore 4. By “engage with” it is meant that friction beads 16 or serrated teeth 45, as described below, on the inner surface of the outer shaft 1 come into physical contact with ridges 15 or serrated teeth 46, as described below, on the inner shaft 20 providing extra resistance against movement of inner shaft 20 through the internal bore 4.

FIGS. 10A-10C show another embodiment of this invention wherein the outer surface of inner shaft 20 contains at least one alignment rib 41 along the length of inner shaft 20. As shown in FIG. 10A, an alignment rib 41 is a section of the outer surface of inner shaft 20 raised higher than the surrounding surface. Serrated teeth 46 extend out from a section of the alignment rib 41.

Also in this embodiment, as shown in FIGS. 11A-11C, the outer shaft 1 has at least one alignment slot 40 cut into its inner surface. The depth, position, and number of alignment slots 40 correspond to the height, position, and number of alignment ribs 41 on inner shaft 20 so that the alignment ribs 41 of inner shaft 20 fit into the alignment slots 40 of the inner surface of outer shaft 1. Serrated teeth 45 extend out from a section of alignment slots 40. The section of alignment slot 40 that contains the serrated teeth 45 corresponds to the section of the alignment rib 41 that contains serrated teeth 46.

In this embodiment, inner shaft 20 fits in the internal bore 4 of the outer shaft 1 when alignment rib 41 is aligned with alignment slot 40. During proximal and distal movement of inner shaft 20 through internal bore 4 of outer shaft 1, the serrated teeth 46 along alignment rib 41 contact and engage with serrated teeth 45 along alignment slot 40 preventing unwanted movement.

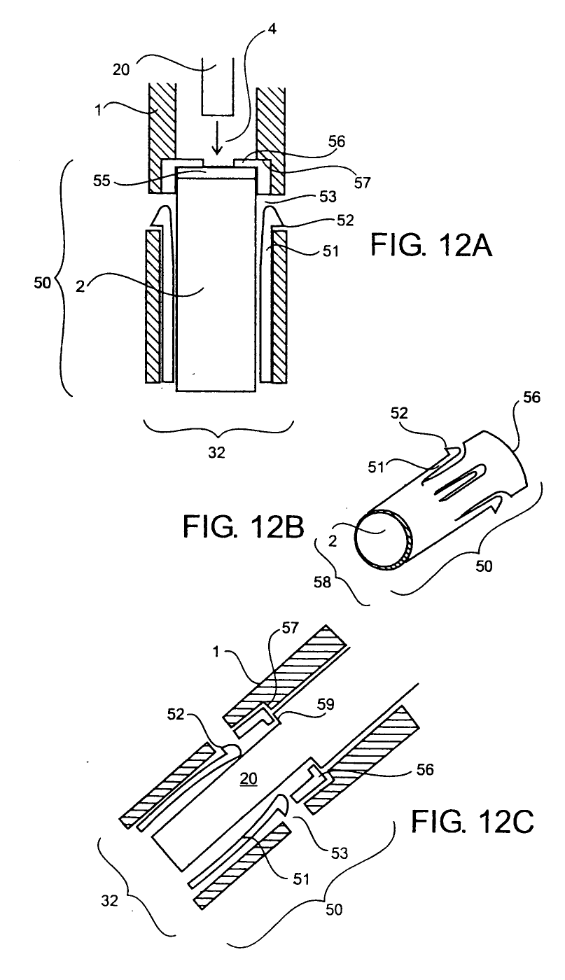

FIGS. 12A-12C illustrate a capsule loader 50 that can be used with implant delivery device 30. The capsule loader 50 is a hollow tube having an outer diameter slightly less than the inner diameter of outer shaft 1 allowing the capsule loader 50 to fit within internal bore 4 at the distal end 32 of outer shaft 1. Optionally, the inner diameter of outer shaft 1 may be decreased along internal bore 4 creating internal shoulder 57. The outer diameter of the capsule loader 50 is great enough that when inserted into outer shaft 1, the capsule loader 50 contacts internal shoulder 57 and is prevented from proximally advancing further through internal bore 4. Preferably internal shoulder 57 is positioned proximally from the distal end 32 of the outer shaft 1 at a distance equal to the length of capsule loader 50 so that when capsule loader 50 contacts internal shoulder 57 the front end 58 of capsule loader 50 is flush with the distal end 32 of the outer shaft 1.

The capsule loader 50 has an inner diameter slightly greater than the diameter of inner shaft 20. The inner diameter of capsule loader 50 is also slightly greater than implant 2, allowing implant 2 to be disposed within capsule loader 50. The back end 56 of capsule loader 50 has a round hole (also called an “opening”) therethrough with a diameter slightly less than the rest of the capsule loader 50 but slightly greater than the diameter of distal end 32 of the inner shaft 20, thus allowing inner shaft 20 to pass through capsule loader 50. Optionally, the diameter of inner shaft 20 is increased at a point proximal from the distal end 32 of the inner shaft 20, preferably at a distance from the distal end 32 of the inner shaft 20 equal to the length of the capsule loader 50, to form shoulder 59. The increased diameter of the inner shaft 20 at shoulder 59 remains less than the inner diameter of the outer shaft 1 but is greater than the diameter of the back end 56 of capsule loader 50. When distally advanced within outer shaft 1, the inner shaft 20 passes through capsule loader 50 until shoulder 59 contacts the back end 56 of capsule loader 50 as shown in FIG. 12C.

The capsule loader 50 contains a backplate 55, which has a diameter slightly less than the inner diameter of the capsule loader 50 allowing it to proximally and distally move through the capsule loader 50. The backplate 55 has a greater diameter than the back end 56 of capsule loader 50. When an implant 2 is disposed within capsule loader 50, the backplate 55 is between implant 2 and the back end 56 of capsule loader 50.

The capsule loader 50 also has at least one flexible leaflet 51. Flexible leaflets 51 are projections on the outer surface of capsule loader 50 that run along the longitudinal axis thereof. Flexible leaflets 51 can be pressed inward but return to their original position when the inward pressure is released. On the ends of the flexible leaflets are prongs 52, which extend outward from capsule loader 50. When the flexible leaflets are not pressed inward, capsule loader 50 cannot be inserted into the outer shaft 1 because prongs 52 do not fit within internal bore 4. When the flexible leaflets 51 are pressed inward, the prongs 52 fit within internal bore 4 of outer shaft 1 and the capsule loader 50 can be inserted.

In conjunction with use of capsule loader 50, there is at least one prong hole 53 cut through outer shaft 1. The dimensions of the prong holes 53 are slightly larger than prongs 52 such that the prongs 52 can fit through prong holes 53. Preferably prong holes 53 are at a distance from the distal end 32 of the outer shaft 1 so that the prongs 52 are aligned with the prong holes 53 when the capsule loader 50 is inserted into outer shaft 1 and the front end 58 is flush with distal end 32 of outer shaft 1.

To use the capsule loader 50 with the implant delivery device 30, the back end 56 of capsule loader 50 with implant 2 already disposed therein is inserted into the distal end 32 of outer shaft 1. To allow the capsule loader 50 to be inserted into internal bore 4, flexible leaflets 51 must be pressed inward. Once the capsule loader 50 is inserted into outer shaft 1 and the inward pressure is released, the flexible leaflets 51 will exert an outward pressure against the inner surface of outer shaft 1. When prongs 52 on the end of flexible leaflets 51 are aligned with prong holes 53 in outer shaft 1, the outer pressure exerted by flexible leaflets 51 will move the prongs 52 into prong holes 53. While prongs 52 are in the prong holes 53, unwanted motion of the capsule loader 50 is prevented. In addition, the capsule loader 50 may be prevented from further proximal movement through internal bore 4 by internal shoulder 57.

Because the diameter of the distal end 32 of inner shaft 20 is slightly less than the diameter of the hole in back end 56 of capsule loader 50, the distal end 32 of inner shaft 20 can be distally advanced through back end 56 and then through capsule loader 50. While distally advancing through capsule loader 50, inner shaft 20 contacts backplate 55 and pushes backplate 55 and implant 2 distally through capsule loader 50. Continued distal movement by inner shaft 20 will push implant 2 out through front end 58 of capsule loader 50 and out through distal end 32 of outer shaft 1 of delivery device 30. When shoulder 59 of inner shaft 20 contacts back end 56 of capsule loader 50, inner shaft 20 cannot be distally advanced further through capsule loader 50.

After implant 2 has been expelled, capsule loader 50 is removed from delivery device 30 by pushing inward on prongs 52 through prong holes 53 while simultaneously pushing inner shaft 20 toward distal end 32. The prongs 52 are pushed out of prong holes 53 and the shoulder 59 of inner shaft 20 will push against the back end 56 of capsule loader 50. Because the prongs 52 no longer hold capsule loader 50 in place, the capsule loader 50 will be pushed out through the distal end 32 of outer shaft 1.

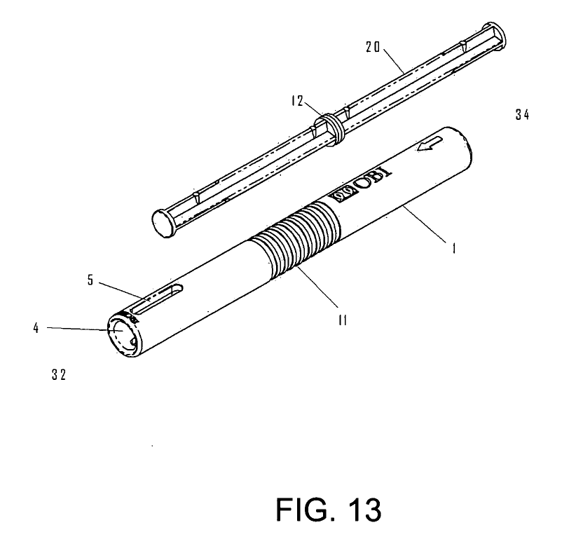

FIG. 13 illustrates one embodiment of the invention where the distal end 32 of the inner 20 shaft and outer shaft 1 are essentially flat. This embodiment is useful when the tissue surrounding the defect is essentially flat and the defect is easily accessible. However, often the surface of the tissue surrounding the defect has a complex surface. In one embodiment, the complex surface comprises an articulating surface. As used herein, a complex surface has a mean curvature that is not constant across the surface. For example, a complex surface is not planar, cylindrical or spherical. Complex surfaces can include, but are not limited to, concave surfaces, convex surfaces (dome-shaped surfaces), saddle-shaped surfaces and other surfaces where, at a given point, the planar curves formed by the intersection of the surface with two orthogonal planes that contain the normal vector to the surface are not uniformly convex or concave, angled surfaces formed by the intersection of two facets, multifaceted domes and multifaceted bowls. In one embodiment, the complex surface has compound radii of curvature, which means that the surface has at least two different (non-infinite) radii of curvature. An implant suitable for the repair of complex surfaces need not be symmetrical. In one embodiment, the implant has one plane of symmetry. Saddle-shaped implants can be used to treat depressed and/or groove areas of joints.

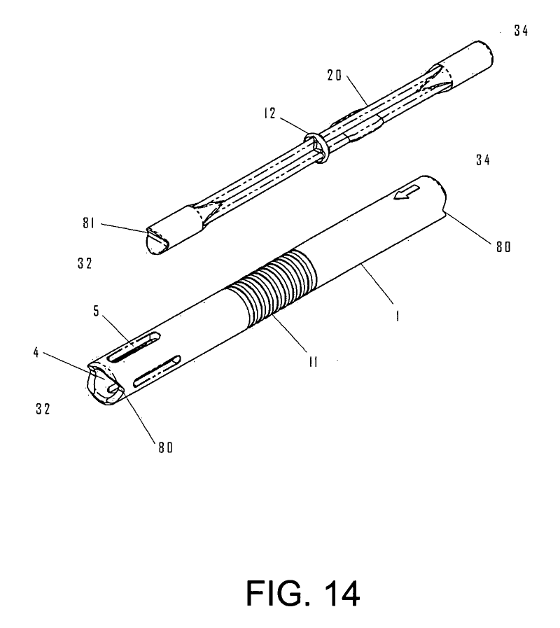

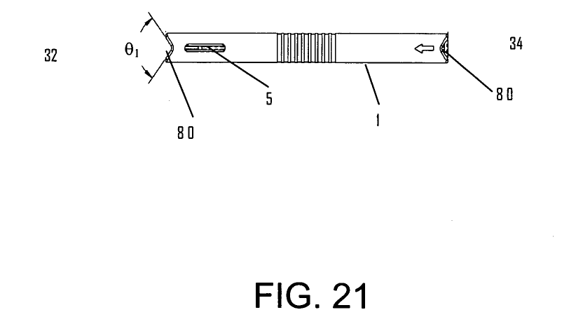

FIG. 14 shows the outer shaft 1 of a delivery device 30 having indentations or notches 80 that are contoured to have a shape corresponding to the tissue surface surrounding the defect (not shown). Both the distal end 32 and proximal end 34 of the outer shaft 1 are optionally shaped to correspond to the shape of the tissue surface. FIG. 21 illustrates the angle, θ₁, of indentation or notch 80 at the distal end 32 of the outer shaft 1. The angles at the distal end 32 and proximal end 34 of the outer shaft 1 are the same, as both ends of the outer shaft 1 will be placed in contact with the complex shape of the tissue surface. The delivery device shown in FIGS. 14 and 21 is suitable for delivery of an implant (not shown) to a defect located on a ridge. For example, if the defect area is on the medial ridge of the talus, θ₁ can be up to about 110 degrees.

The delivery device 30 illustrated in FIG. 14 further comprises an inner shaft 20 also having a distal end 32 and proximal end 34. In use, the inner shaft 20 is situated within the outer shaft 1 and is able to move proximally and distally through the internal bore 4. The distal end 32 of the inner shaft 20 is shaped to correspond to the proximal surface of the implant (not shown). In FIG. 14, the distal end 32 of the inner shaft 20 has an indentation or notch, referred to as tamp indentation 81. For the delivery device in FIG. 14, the notch angle and/or shape of tamp indentation 81 is the same as the angle formed by the proximal end of the implant. The distal and proximal ends of the delivery device may be shaped differently than shown in FIG. 14. For example, for an implant with a concave shape, the implant delivery device would have convex proximal and distal ends for matching the anatomical geometry of the articular surface.

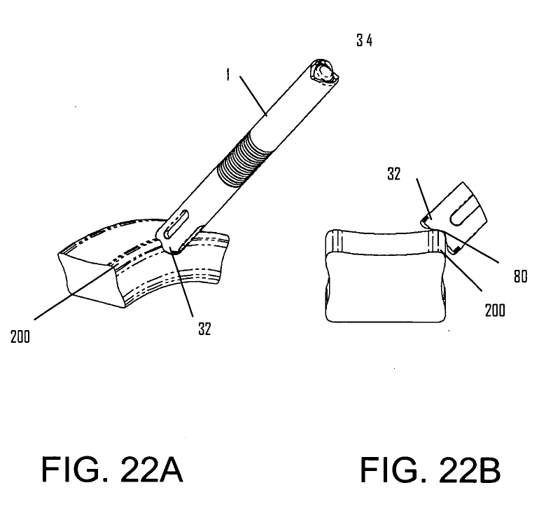

FIGS. 22A and 22B show the outer shaft 1 in contact with ridged tissue surface 200. Indentation or notch 80 in the distal end 32 of the outer shaft 1 contacts the tissue surface 200. In use, the outer shaft 1 is oriented with respect to the tissue so that the proximal or distal end of the outer shaft 1 effectively conforms to the surface of the tissue surrounding the defect. Since indentation or notches 80 at the proximal end 34 and distal end 32 of the outer shaft 1 have been shaped to correspond to the shape of the tissue surrounding the defect, the outer shaft 1 is oriented to maximize contact between the proximal or distal end of the outer shaft and the tissue surrounding the defect.

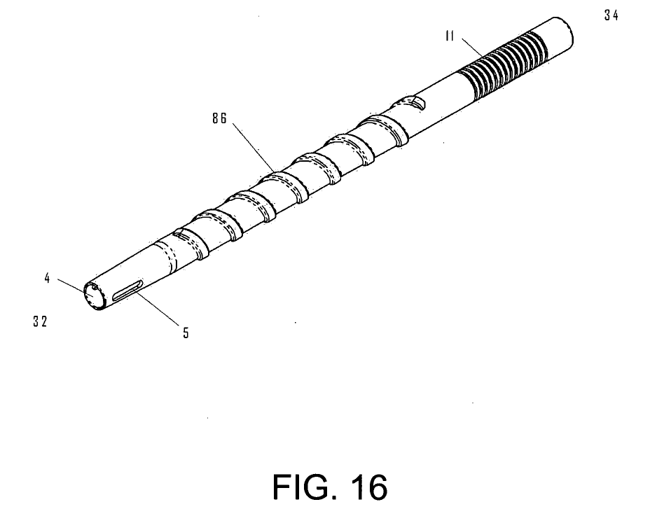

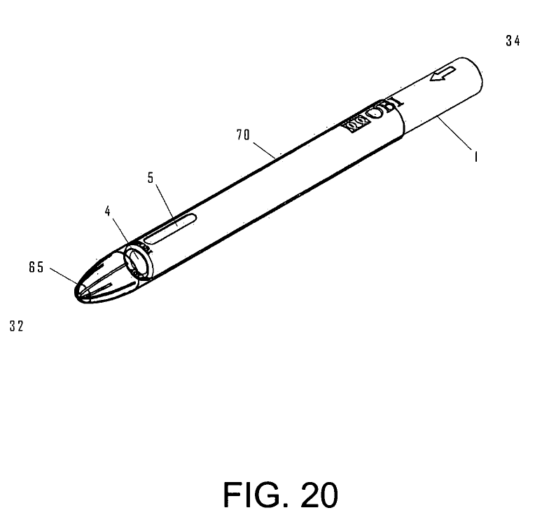

FIG. 16 shows a delivery device of the present invention comprising spiral threading 86 along the length of the outer shaft 1. The threading 86 on the outer shaft 1 is similar to the threads on a screw. When the delivery device 30 is twisted in the appropriate direction, the threading 86 will advance the delivery device through the soft tissue. The threading 86 allows the delivery device to pass through the soft tissue more easily, regardless of whether it is the distal end 32 or the proximal end 34 of the delivery device 30 that is being introduced to the defect. FIG. 20 shows a removable sleeve 70 disposed over the outer shaft 1 of a delivery device. Preferably, the removable sleeve 70 has a bullet-shaped tip 65 so that the outer shaft can be pushed through the soft tissue.

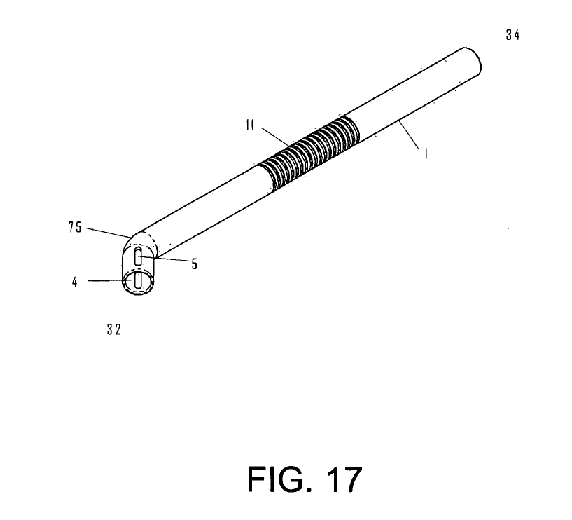

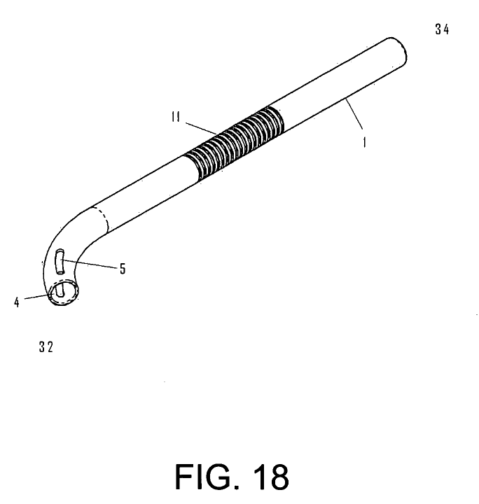

FIG. 17 shows a delivery device where the outer shaft 1 is bent at or near the distal end of the outer shaft 1. By “bent”, it is meant that the outer shaft 1 is not straight but forms an angle between the distal end 32 and the rest of the outer shaft 1. The angle forms a corner 75 and can be any degree between 0 and 180 degrees, more preferably between 10 and 90 degrees, that still allows the inner shaft (not shown) to advance through the inner bore (not shown) to push the implant into the defect. The angle allows the delivery device 30 to approach the defect from a wide range of different angles and positions instead of just perpendicular to the tissue surrounding the defect. Instead of forming a potentially abrupt corner 75, the outer shaft 1 is optionally curved at or near the distal end 32 of the outer shaft 1, as is shown in FIG. 18. The curved tip also forms an angle between the distal end 32 and the rest of the outer shaft 1, and provides a more gradual and easier path for the inner shaft (not shown) to travel through the internal bore (not shown) toward the distal end 32.

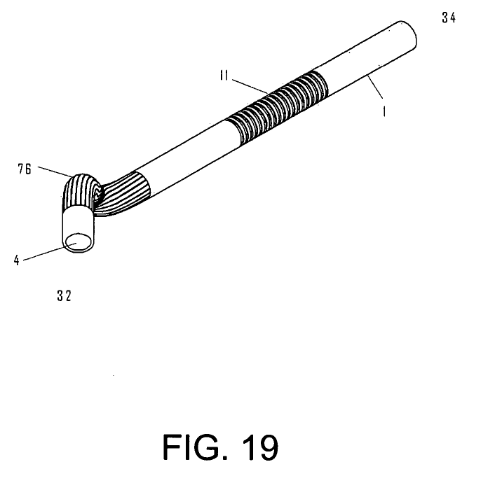

Each defect may require a different angle of the distal end of the outer shaft in order to efficiently and accurately deliver the implant. It would be convenient if the same delivery device could be used to deliver implants to different types and positions of defects. In one such embodiment, as illustrated in FIG. 19, a delivery device 30 of the invention comprises a flexible section 76 at or near the distal end 32 of said outer shaft 1, which allows the distal end 32 to be bent or shaped into different angles and positions. The flexible section 76 is flexible enough so that the angle and orientation of the distal end can be adjusted by hand, but is able to retain its shape while the delivery device is being positioned over the defect. The position and angle of the distal end 32 of the outer shaft 1 is adjusted for every implant delivery to the position and angle necessary to deliver the implant to the defect.

The flexible section can a hinge or a plethora of hinges that allow the delivery device 30 to change its angle along the body. Alternatively, a low-modulus polymer or elastomer can be used to create an articulating section along the body of the device. At some point between the proximal end and distal end (more preferably located nearer the distal end, close to the implant-containing section) the rigid outer shaft of the device is interrupted by a semi-rigid section of bendable material. This section is sufficiently rigid to prevent undesired flexion upon insertion (i.e. through soft tissue in an arthoscopic procedure), yet may be bent by applying a moment about the flexible section. The inner shaft can also be made flexible in a similar manner, or may be provided as a flexible section along the entire length of the component. The flexible section(s) may be further reinforced by incorporating radially distributed structural elements, such as wires, within the low modulus material. For example, a flexible section within the delivery device may be created by disposing a section of medium durometer silicon (e.g. 60 shore A) with a central bore that is contiguous with the bore of the delivery device between two rigid sections of the delivery device. The flexible section can be color matched to the material used for the rigid sections, or could be clear, opaque or colored differently (i.e. black). The flexible section can be further reinforced by disposing a spirally wound wire within the elastomer to provide for additional radial support during flexion. The flexible section may be further improved by making it in the form of a bellows which will allow it to flexibly deform with reduced bending force.

The inner shaft disposed within the outer shaft can have a similar flexible section as described above (with or without an internal bore or lumen), or may be made continuously flexible, for example by incorporating a spirally wound spring with good compressive strength but with reduced flexural resistance.

All patents and publications mentioned in the specification are indicative of the levels of skill of those skilled in the art to which the invention pertains. One skilled in the art would readily appreciate that the present invention is well adapted to carry out the objects and obtain the ends and advantages mentioned, as well as those inherent therein. The devices, methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims.

When a Markush group or other grouping is used herein, all individual members of the group and all combinations and subcombinations possible of the group are intended to be individually included in the disclosure.

While the invention has been described with certain preferred embodiments, it is understood that the preceding description is not intended to limit the scope of the invention. It will be appreciated by one skilled in the art that various equivalents and modifications can be made to the invention shown in the specific embodiments without departing from the spirit and scope of the invention. All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis, and additional uses of the invention.

We claim:

1. A bone or cartilage implant delivery device comprising:
a tubular outer shaft having a proximal and distal end, a longitudinal axis, and an internal bore along the longitudinal axis of said outer shaft;
an inner shaft having a distal end and a proximal end suitable for insertion into a defect, said inner shaft adapted to fit within said internal bore of the outer shaft so that the inner shaft and the outer shaft are slidably engaged.

2. The device of claim 1 wherein the distal end of the outer shaft is at least partially translucent or transparent.

3. The device of claim 2 wherein the entire outer shaft is translucent or transparent.

4. The device of claim 3 wherein the outer shaft is transparent.

5. The device of claim 3 wherein the inner shaft is translucent or transparent.

6. The device of claim 5 wherein the outer shaft or inner shaft is translucent and tinted with a color.

7. The device of claim 3 wherein the inner shaft is opaque and colored.

8. The device of claim 1 wherein the device is constructed from medical grade plastic or metal.

9. The device of claim 1 wherein the outer shaft is bent at or near the distal end of the outer shaft.

10. The device of claim 9 wherein the distal end of the outer shaft forms an angle up to 90 degrees with the rest of the outer shaft.

11. The device of claim 9 wherein the outer shaft is curved at or near the distal end of the outer shaft.

12. The device of claim 1 wherein the outer shaft comprises a flexible section at or near the distal end of said outer shaft, where said flexible section allows the distal end to be bent or shaped into different angles and positions.

13. The device of claim 1 further comprising a removable sleeve having a bulleted tip, where said removable sleeve is able to fit over the distal end or proximal end of the outer shaft.

14. The device of claim 13 wherein said removable sleeve is transparent.

15. The device of claim 1 wherein the distal end of the outer shaft is tapered inward.

16. The device of claim 1 wherein the device is between 4 and 10 inches long, with the internal bore having a diameter between 0.2 and 1.0 inches.

17. The device of claim 1 further comprising spiral threading along the length of the outer shaft.

18. The device of claim 1 wherein the inner shaft has a cannula through its center.

19. The device of claim 18 further comprising a guide wire disposed in said cannula, where one end of said guide wire is attached to a defect and the other end of the guide wire passes through the distal end of the inner shaft and extends to the proximal end of the inner shaft.

20. The device of claim 1 further comprising notches at the distal end and proximal end of the outer shaft that conform to the surface of the tissue surrounding the defect.

21. The device of claim 1 further comprising an implant having a proximal surface that matches the contours of the tissue surrounding the defect, and a notch at the distal end of the inner shaft that conforms to the proximal surface of the implant.

22. A method for delivering a bone or cartilage implant into a defect in a tissue having an unmeasured depth using the implant delivery device of claim 1 comprising the steps:
inserting said implant into the distal end of said loading device, wherein when said implant is disposed in said loading device the proximal end of the inner shaft protrudes from the proximal end of the outer shaft and the length of said implant and equals the length of the protruding section of the inner shaft;
inserting the proximal end of the inner shaft into the defect until the proximal end of the inner shaft contacts the bottom of the defect;
advancing the outer shaft in the proximal direction until the proximal end of the outer shaft contacts the surface of tissue surrounding the defect, causing a portion of the implant to extend beyond the distal end of the outer shaft;
cutting off the portion of the implant extending beyond the distal end of the outer shaft, leaving a remaining portion disposed within the outer shaft;
placing the distal end of the loading device over the defect, visualizing the implant in the distal end of the outer shaft, and orientating the device so that the implant is in the desired position in relation to the defect; and
distally advancing the inner shaft to push the portion of the implant remaining after cutting into the defect.

23. The method of claim 22 wherein at least part of the distal end of the outer shaft is transparent or translucent.

24. The method of claim 22 wherein the outer shaft is bent or curved at or near the distal end of the outer shaft.

25. The method of claim 24 wherein the distal end of the delivery device is placed over the defect by advancing the delivery device at an angle other than perpendicular to the tissue surrounding the defect.

26. A kit comprising at least one bone or cartilage implant delivery device, said implant delivery device comprising:
a tubular outer shaft having a proximal and distal end, a longitudinal axis, and an internal bore along the longitudinal axis of said outer shaft, wherein the outer shaft is translucent or transparent; and
an inner shaft having a distal end and a proximal end suitable for insertion into a defect, said inner shaft adapted to fit within said internal bore of the outer shaft so that the inner shaft and the outer shaft are slidably engaged.

27. The kit of claim 26 further comprising an implant.

28. The kit of claim 26 comprising a plurality of bone or cartilage implant delivery devices each having different sizes of internal bores and inner shafts.

29. The kit of claim 28 wherein the inner shaft of each delivery device is opaque and has a color corresponding to the size of the internal bore and inner shaft of the delivery device.

30. The kit of claim 29 wherein the inner shaft is translucent or transparent and the outer shaft or inner shaft is tinted with a color corresponding to the size of the internal bore and inner shaft of the delivery device.
